(19) United States
(12) Patent Application Publication
(10) Pub. No.: US 2015/0359898 A1
(43) Pub. Date: Dec. 17, 2015

(54) Pemetrexed Complexes and Pharmaceutical Compositions Containing Pemetrexed Complexes

(71) Applicant: CIPLA LIMITED, Mumbai (IN)

(72) Inventors: Shrinivas Madhukar Purandare, Mumbai, Maharashtra (IN); Geena Malhotra, Maharashtra, Mumbai (IN); Dharmaraj Ramachandra Rao, Maharashtra, Mumbai (IN); Rajendra Narayanrao Kankan, Maharashtra, Mumbai (IN); Srinivas Laxminarayan Pathi, Karnataka, Bangalore (IN); Ravikumar Puppla, Karnataka, Bangalore (IN)

(73) Assignee: CIPLA LIMITED, Mumbai (IN)

(21) Appl. No.: 14/765,920

(22) PCT Filed: Feb. 6, 2014
(86) PCT No.: PCT/GB2014/050340

§ 371 Date: Aug. 5, 2015

(30) Foreign Application Priority Data
Feb. 6, 2013 (IN) 355/MUM/2013
Feb. 7, 2013 (IN) 368/MUM/2013

(51) Int. Cl. A61K 47/48; A61K 31/519; C07H 3/04; C07D 487/04; C07C 31/26

(52) U.S. Cl. CPC A61K 47/48023; C07D 487/04; C07C 31/26; C07H 3/04; A61K 47/48092; A61K 31/519; C07B 2200/13

(57) ABSTRACT

A complex comprises pemetrexed and a coformer. A pharmaceutical composition comprises a complex of pemetrexed and a co-former and one or more pharmaceutically acceptable excipients.

The present invention relates to pharmaceutically acceptable complexes comprising pemetrexed and pharmaceutically acceptable compositions comprising said complexes, processes for preparation of the complexes and pharmaceutical compositions comprising said complexes, and their use in the treatment of cancer, pleural mesothelima and non-small cell lung cancer.

### BACKGROUND OF THE INVENTION

Pyrrolo[2,3-d]pyrimidine based antifolates are drugs that impair the function of folic acids. Many of them are used in cancer chemotherapy and some are used as antibiotics or antiprotozoal agents. These include pemetrexed, methotrexate, raltitrexed and pralatrexate.

Pemetrexed is an antifolate, antineoplastic agent. Lily's anticancer drug Alimta® (pemetrexed) is a sterilized lyophilized powder for intravenous administration and is approved for treatment of mesothelioma and non small cell lung cancer.

Pemetrexed was approved by the United States Food and Drug Administration in February 2004 in combination with cisplatin, a platinum-containing chemotherapeutic drug for the treatment of malignant pleural mesothelioma (MPM). In July 2004, the drug was approved by the FDA as a second line agent for the initial treatment of advanced or metastatic non-small cell lung cancer (NSCLC). Currently, the drug is used as a single agent or in combination with other chemotherapeutic agents for the treatment of other types of cancer such as breast cancer, bladder cancer, colorectal carcinoma and cervical cancer.

Pemetrexed was first disclosed in U.S. Pat. No. 5,344,932 and is commercialized as Alimta sterile lyophilized powders for intravenous infusion, available in single-dose vials containing 100 mg or 500 mg equivalent of pemetrexed.

A crystalline form of pemetrexed disodium designated Disodium MTA Hydrate Form I has been described in WO200114379.

U.S. Pat. No. 7,138,521 describes a stable crystalline heptahydrate form of pemetrexed having a characteristic X-ray diffraction pattern, which comprises a peak corresponding to a d-spacing of 7.78±0.04 Å when obtained at 22±2° C. and at ambient relative humidity. The patent mentions that the heptahydrate form is much more stable than the previously known 2.5 hydrate and the primary advantage over the 2.5 hydrate crystal form is its stability and also with respect to the formation of related substances. The patent also indicates that when the heptahydrate is subjected to elevated temperatures, low humidity, and/or a vacuum, it converts to the 2.5 hydrate crystal form by loss of water.

WO2008124485 discloses besides five crystalline forms of the diacid pemetrexed; an amorphous pemetrexed disodium as well as a crystalline Form III thereof including a composition containing a major amount of amorphous Form and a minor amount of crystalline Form III of pemetrexed disodium.

EP1943252 discloses a process for the preparation of a lyophilized pharmaceutically acceptable di-base-addition salt of pemetrexed, in particular, a pemetrexed disodium salt, directly from pemetrexed diacid or an acid or base addition salt thereof, i.e. a di-base salt of pemetrexed which is not isolated prior to the lyophilization.

US2011201631A1 discloses a solid pharmaceutical formulation comprising amorphous pemetrexed, or a salt thereof, and at least one pharmaceutically acceptable excipient.

The crystalline, amorphous and hydrate forms of pemetrexed have been disclosed through different prior art patent, applications and literature. The crystalline heptahydrate forms, 2.5 hydrate form have also been disclosed and pharmaceutical composition comprising these different forms of pemetrexed are known through prior art.

Prior art discloses various pharmaceutical compositions comprising different forms and salts of pemetrexed.

Properties such as the solubility and stability and consequently the suitability for use and storage of each solid form may vary and is essential for providing pharmaceuticals with increased storage stability or predicable solubility profiles. Each crystalline form of a drug candidate can have different solid-state (physical and chemical) properties which may be relevant for drug delivery.

Dissolution rates of an active ingredient in vivo (e.g., gastric or intestinal fluid) may have therapeutic consequences since it affects the rate at which an orally administered active ingredient may reach the patient's bloodstream. In addition, solubility, a thermodynamic quantity, is a relevant property in evaluating drug delivery because a poorly soluble crystalline form of a drug will deliver less drug than a more soluble one in the same formulation.

Because these practical physical properties are influenced by the solid-state properties of the crystalline form of the drug, they can significantly impact the selection of a compound as a drug, the ultimate pharmaceutical dosage form, the optimization of manufacturing processes and absorption in the body. Moreover, finding the most adequate solid state form for further drug development can reduce the time and the cost of development.

However, the conversions between different polymorphic forms of pemetrexed when exposed to elevated temperatures, low humidity etc. may affect the formulation processes, resulting in a possibility that the stability of the final product may be affected. It is known that amorphous forms of active ingredients can be relatively more unstable, when compared to the crystalline form. Thus, stabilizing amorphous pemetrexed or its salts in formulations is considered difficult. Hence, formulating pemetrexed has not proven to be an easy task, due to its stability issues.

Therefore, obtaining a suitable form of a drug is a necessary stage for many drug substances. Also, molecular and thermodynamic properties of the drug form contribute to the solubility and stability of a drug product for its pharmaceutical use.

Additionally, while designing any pharmaceutical composition it is important to lay emphasis on the API and excipients. The selection of the API type and excipients would ultimate decide the fate of bioavailability and robustness of the ultimate composition. The excipients API compatibility is of utmost importance to design a stable, bioavailable and robust composition. Secondly the form conversion of the API during the pharmaceutical composition processing due to change in temperature or humidity of the environment also needs to be considered.

The inventors of the present invention have appreciated that prior art forms of pemetrexed and pharmaceutical compositions comprising such forms of pemetrexed could be improved by tailoring physical properties such as stability of the API and solubility of the pharmaceutical compositions. However, there is a need to do this without affecting the chemical composition of the API. The inventors have appreciated that these problems could be solved by providing complexes formed by the pemetrexed API and a co-former or molecular complexes. These can be tailored to address formulation issues related to the physical properties such as solubility and stability without affecting the chemical composition of the API. These complexes can be further incorporated into the compositions to design stable and robust pharmaceutical compositions.

### OBJECTS OF THE INVENTION

An object of the present invention is to provide pharmaceutically acceptable complexes of pemetrexed.

Another object of the present invention is to provide a pharmaceutical composition comprising complexes of pemetrexed along with one or more pharmaceutically acceptable excipients.

Another aspect of the invention is to provide pharmaceutical composition comprising complexes of pemetrexed which are stable and bioavailable.

Yet object of the invention is to provide process of preparation of pharmaceutical composition comprising complexes of pemetrexed along with one or more pharmaceutically acceptable excipients.

Another object of the invention is to provide a method of treating cancer, pleural mesothelima and non-small cell lung cancer by administering a pharmaceutical composition comprising complexes of pemetrexed along with one or more pharmaceutically acceptable excipients.

Another object of the present invention is to provide a pharmaceutical composition comprising complexes of pemetrexed along with one or more pharmaceutically acceptable excipients for use in the treatment of cancer, pleural mesothelima and non-small cell lung cancer.

### SUMMARY OF THE INVENTION

According to an aspect of the invention, there is provided a complex of pemetrexed with a co-former.

According to another aspect of the invention, there is a provided a complex of the invention for use in medicine. Preferably, the use comprises the treatment of cancer, pleural mesothelima and/or non-small cell lung cancer.

According to another aspect of the invention, there is provided use of a complex of the invention in the manufacture of a medicament for the treatment of cancer, pleural mesothelima and/or non-small cell lung cancer.

According to another aspect of the invention, there is provided a method of treating a subject with cancer, pleural mesothelima and/or non-small cell lung cancer, wherein the method comprises administering a complex according to the present invention to a patient in need thereof.

According to another aspect of the invention, there is provided a process for the preparation of a complex of the invention, wherein the process comprises;

(1) dispersing pemetrexed into an aqueous solvent to form a solution;

and adjusting the pH of the solution;

(2) optionally removing insolubles by filtration;

(3) adding a carbohydrate to the filtrate;

(4) precipitating and drying the complex from the solution.

According to another aspect of the invention, there is provided a pharmaceutical composition comprising a complex of pemetrexed, and one or more pharmaceutically acceptable excipients.

According to another aspect of the invention, there is provided a pharmaceutical composition of the invention for use in medicine. Preferably, the use comprises the treatment of cancer, pleural mesothelima and/or non-small cell lung cancer.

According to another aspect of the invention, there is provided use of a pharmaceutical composition of the invention in the manufacture of a medicament for the treatment of cancer, pleural mesothelima and/or non-small cell lung cancer.

According to another aspect of the invention, there is provided a method of treating a subject with cancer, pleural mesothelima and/or non-small cell lung cancer, wherein the method comprises administering a complex according to the present invention to a patient in need thereof.

According to another aspect of the present invention, there is provided a process of preparing a pharmaceutical composition according to the invention, wherein the process comprises dispersing a complex of pemetrexed optionally with at least one or more pharmaceutically acceptable excipients, adjusting the pH using a suitable pH adjusting agent; optionally filling the mixture into a container; and optionally lyophilising the mixture.

### DETAILED DESCRIPTION

The present invention provides a complex comprising pemetrexed and at least one co-former and methods of preparing such complexes. The present invention also provides pharmaceutical compositions comprising pemetrexed complexes with one or more pharmaceutically acceptable excipients. Preferably, pemetrexed is in the form of pemetrexed disodium or pemetrexed dipotassium or pemetrexed with any other metal salts. More preferably, pemetrexed is in the form of pemetrexed disodium.

The term “Pemetrexed” is used in broad sense to include not only “Pemetrexed” per se but also its pharmaceutically acceptable derivatives thereof. Suitable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

A “complex” according to the present invention is a single chemical entity comprising two or more different elements that have a unique and defined chemical structure. Complexes can be constructed through several modes of molecular recognition including hydrogen-bonding, z (pi)-stacking, guest-host complexation and Van Der Waals interactions. Of the interactions listed above, hydrogen-bonding is the dominant interaction in the formation of the pharmaceutical compounds, whereby a non-covalent bond is formed between a hydrogen bond donor of one of the moieties and a hydrogen bond acceptor of the other. A complex of the present invention is considered to be one where hydrogen bonding occurs between pemetrexed and a co-former. A co-crystal is a crystalline structure composed of at least two components, where the components may be atoms, ions or molecules. In the present disclosure, the co-crystal is a crystalline structure composed of at least two molecules; suitably two molecules.

The pharmaceutically acceptable complex of pemetrexed disodium or pemetrexed dipotassium used in the compositions of the present invention is a single chemical entity comprising two or more different elements that have a unique and defined chemical structure. Pemetrexed Complex used in the pharmaceutical compositions of the present invention involves hydrogen bonding between pemetrexed and a co-former.

Preferably, the complex of the present invention is a complex of pemetrexed and at least one co-former which is optionally a crystal co-forming agent. Preferably, the cofomer is a carbohydrate.

Preferably, the complex of the invention is a complex of pemtrexed disodium or pemetrexed dipotassium and a carbohydrate. Preferably, the carbohydrate is a sugar and/or sugar alcohol.

The carbohydrate may be a sugar selected from the group comprising of glucose, dextrose, fructose, galactose, ribose, sucrose, xylose, trehalose, lactose, maltose, raffinose, melezitose, glycerol, mannitol, sorbitol, erythritol, xylitol, maltitol, lactitol, D series and L series of rare sugars.

The D series of rare sugars are selected from D-sorbose, D-psicose, D-tagatose, D-gulose, D-gulitol, D-idose, D-iditol, D-talose, D-talitol, D-galactitol, D-mannitol, D-glucitol, D-altrose, D-altritol, D-allose and D-allitol.

The L series of rare sugars are selected from L-sorbose, L-fructose, L-psicose, L-tagatose, L-gulose, L-gulitol, L-idose, L-iditol, L-talose, L-talitol, L-galactitol, L-galactose, L-mannitol, L-mannose, L-glucitol, L-glucose, L-altrose, L-altritol, L-allose and L-allitol.

Preferably, the complex of the present invention is amorphous or crystalline in nature. Preferably, the complex is a co-crystal.

The components of the complex which are pemetrexed disodium or pemetrexed dipotassium and carbohydrate can be present in a substantially stoichiometric ratio. Preferably, the complex of pemetrexed disodium or pemetrexed dipotassium and carbohydrates comprise carbohydrates in an amount of from about 5% to about 50% by weight of the complex.

Preferably, the complex is pemetrexed disodium:mannitol complex, pemetrexed disodium:sorbitol complex, pemetrexed disodium:sucrose complex, pemetrexed disodium:glucose complex, or pemetrexed disodium:fructose complex.

Preferably, the complex is pemetrexed dipotassium:mannitol complex, pemetrexed dipotassium:sorbitol complex, pemetrexed dipotassium:sucrose complex, pemetrexed dipotassium:glucose complex, or pemetrexed dipotassium:fructose complex.

The pemetrexed disodium:mannitol complex comprises pemetrexed disodium and mannitol in an almost stoichiometric ratio. The pemetrexed dipotassium:mannitol complex comprises pemetrexed dipotassium and mannitol in an almost stoichiometric ratio. The content of mannitol in the complex may be in the range of about 20% to about 50%, preferably from about 25% to about 45% or more preferably from about 30% to about 40%.

The crystalline nature of pemetrexed disodium:mannitol complex has been analyzed, characterized and differentiated by X-ray powder diffraction, a technique which is well known per se.

The X-ray powder diffraction pattern was measured on a Rigaku Dmax 2200 advanced X-ray powder diffractometer with a copper-K-a radiation source.

In an embodiment, the crystalline complex of pemetrexed disodium:mannitol has an XRD pattern comprising peaks at 9.619, 20.299 and 22.82 °2θ±0.2 °2θ. The XRD pattern may further comprise peaks at 15.44, 20.98, 21.98, 23.8, 24.559, 25.16 and 36.00 2θ±0.2 °2θ. The XRD pattern may comprise still further peaks at 4.66, 10.38, 14.12, 15.719, 17.14, 17.90, 18.559, 19.261, 25.861, 26.90, 27.78 and 28.82 °2θ±0.2 °2θ. Thus, typically, the complex of pemetrexed disodium:mannitol has an XRD pattern comprising peaks at 4.66, 9.619, 10.38, 14.12, 15.44, 15.719, 17.14, 17.90, 18.559, 19.261, 20.299, 20.98, 21.98, 22.82, 23.8, 24.559, 25.16, 25.861, 26.90, 27.78, 28.82 and 36 °2θ±0.2 °2θ.

The complex is in a substantially pure form; preferably substantially free from other forms. The X-ray diffraction pattern of the pure form does not show any diffraction peaks that allow calculation of a d-spacing of about 7.78±0.04 Å, corresponding to 11.3 °2θ.

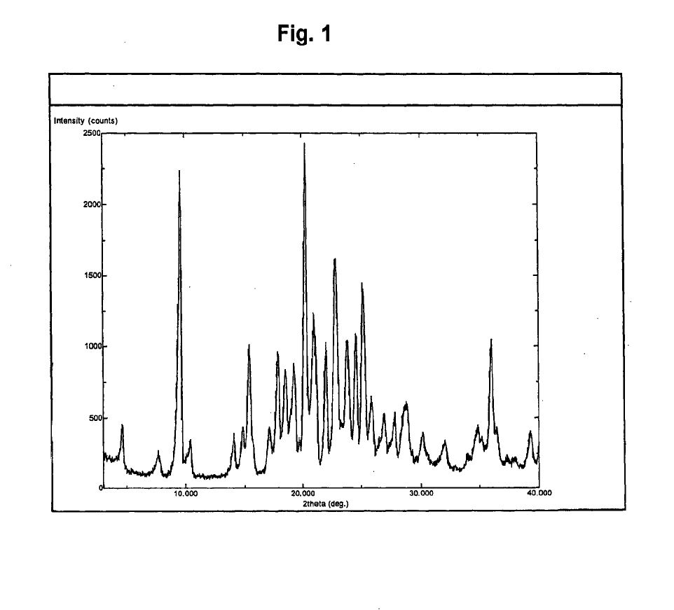

In an embodiment, the pemetrexed disodium:mannitol complex is characterized by having an X-ray powder diffraction spectrum as shown in FIG. 1. The XRD peaks of the pemetrexed disodium:mannitol complex are identified in Table 1 below

| Diffraction angle (2θ-values) | Intensity (%) |
|---|---|
| 4.660 | 14.6 |
| 7.700 | 7.1 |
| 9.619 | 100.0 |
| 10.380 | 11.2 |
| 14.120 | 11.1 |
| 14.879 | 8.9 |
| 15.440 | 43.6 |
| 15.719 | 11.8 |
| 17.140 | 15.8 |
| 17.900 | 29.8 |
| 18.559 | 19.7 |
| 19.261 | 25.5 |
| 19.716 | 2.7 |
| 20.299 | 94.1 |
| 20.980 | 51.0 |
| 21.980 | 38.2 |
| 22.820 | 63.8 |
| 23.800 | 31.6 |
| 24.559 | 37.1 |
| 25.160 | 52.4 |
| 25.861 | 15.5 |
| 26.900 | 11.8 |
| 27.780 | 12.0 |
| 28.820 | 18.6 |
| 30.160 | 9.8 |
| 32.040 | 8.1 |
| 33.938 | 1.6 |
| 34.840 | 9.0 |
| 35.180 | 3.3 |
| 36.000 | 36.7 |
| 36.400 | 12.0 |
| 37.262 | 1.7 |
| 39.359 | 10.5 |

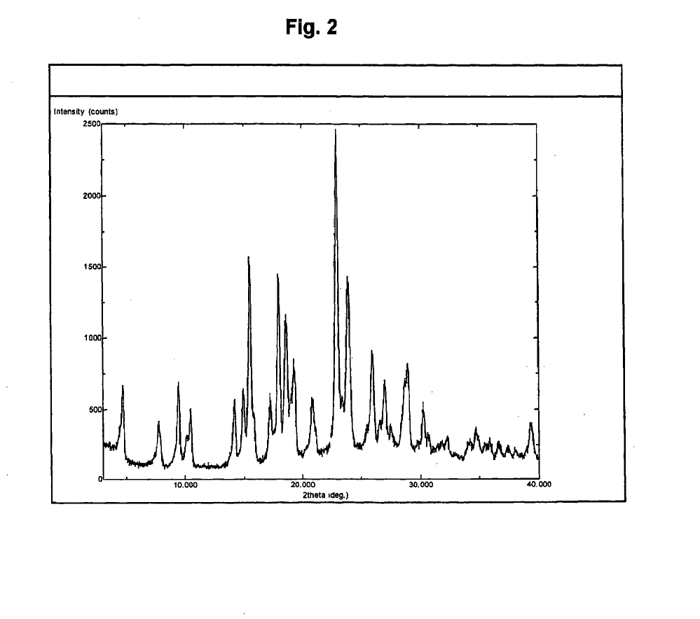

In another embodiment, the pemetrexed disodium:sorbitol complex is characterized by having an X-ray powder diffraction spectrum as shown in FIG. 2. The XRD peaks of the pemetrexed disodium:sorbitol complex are identified in Table 2 below.

| Diffraction angle (2θ-values) | Intensity (%) |
|---|---|
| 4.720 | 25.0 |
| 7.760 | 13.1 |
| 9.459 | 24.7 |
| 10.140 | 8.9 |
| 10.460 | 18.6 |
| 14.200 | 18.0 |
| 14.939 | 16.2 |
| 15.520 | 66.7 |
| 15.800 | 16.0 |
| 17.221 | 20.9 |
| 17.980 | 50.4 |
| 18.620 | 33.6 |
| 19.040 | 18.9 |
| 19.299 | 30.2 |
| 20.839 | 17.9 |
| 22.920 | 100.0 |
| 23.362 | 2.6 |
| 23.860 | 55.1 |
| 25.481 | 4.4 |
| 25.960 | 28.3 |
| 26.580 | 5.6 |
| 26.981 | 18.2 |
| 27.500 | 4.6 |
| 28.681 | 21.3 |
| 28.920 | 26.1 |
| 29.793 | 1.3 |
| 30.240 | 14.0 |

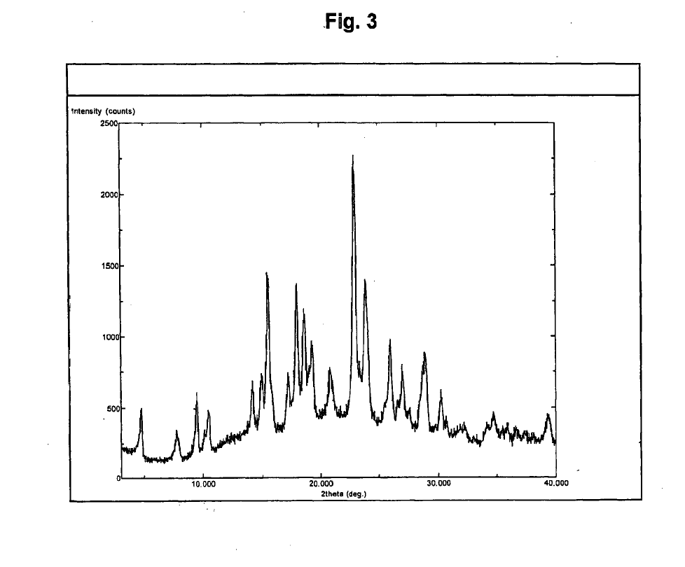

In still another embodiment, the pemetrexed disodium:sucrose complex is characterized by having an X-ray powder diffraction spectrum as shown in FIG. 3. The XRD peaks of the pemetrexed disodium:sucrose complex are identified in Table 3 below.

| Diffraction angle (2θ-values) | Intensity (%) |
|---|---|
| 4.741 | 20.0 |
| 7.780 | 11.1 |
| 9.460 | 23.4 |
| 10.121 | 6.9 |
| 10.460 | 16.6 |
| 13.839 | 3.4 |
| 14.220 | 17.8 |
| 14.959 | 14.2 |
| 15.540 | 58.6 |
| 15.838 | 11.1 |
| 17.239 | 13.9 |
| 17.981 | 46.0 |
| 18.640 | 31.9 |
| 19.060 | 13.2 |
| 19.281 | 22.7 |
| 20.821 | 19.1 |
| 22.921 | 100.0 |
| 23.362 | 3.5 |
| 23.880 | 47.4 |
| 25.519 | 5.4 |
| 25.980 | 30.4 |
| 26.600 | 6.3 |
| 26.961 | 19.2 |
| 27.520 | 3.6 |
| 28.403 | 7.3 |
| 28.740 | 26.0 |
| 28.939 | 30.2 |
| 30.260 | 15.1 |
| 30.680 | 4.5 |

According to another aspect of the invention, there is provided a process for the preparation of a complex, wherein the process comprises;

(1) dispersing pemetrexed into an aqueous solvent to form a solution;

and adjusting the pH of the solution;

(2) optionally removing insolubles by filtration;

(3) adding a carbohydrate to the filtrate;

(4) precipitating and drying the complex from the solution.

The pH may be adjusted to 8 with aqueous sodium hydroxide solution.

After a carbohydrate is added to the filtrate, a clear solution may be obtained.

The process may be carried out under an inert atmosphere, and may be carried out at a temperature ranging from 20° C. to 30° C.

The pemetrexed used as a starting material may be in any form. For example the pemetrexed may be a free acid or in a polymorphic form, in a mixture of polymorphic forms, crude or in anhydrous, hydrated or solvated form and the like.

The carbohydrate may be a sugar and/or sugar alcohol which may be selected from the sugars described hereinbefore.

The pemetrexed disodium:carbohydrate or pemetrexed dipotassium:carbohydrate complex may be precipitated by adding an antisolvent. The antisolvent may be a water-miscible solvent. The antisolvent may be selected form the group consisting of, but not limited to, acetone, C1-6 alcohols, and acetonitrile. In one embodiment, acetone is used as an anti-solvent and the like or mixtures thereof. The C1-6 alcohols may include isopropyl alcohol.

The precipitated crystals are collected by filtration and may be dried at an elevated temperature ranging from about 30° C. to about 60° C., and preferably ranging from about 40° C. to about 50° C.

The process involves isolation of the complex in a solvent-free medium, thus producing a complex which is free of solvents or having a negligible solvent content.

According to the present invention, there is provided a pharmaceutical composition comprising the complex of the present invention, with one or more pharmaceutically acceptable excipients. The complexes used in the pharmaceutical compositions can be any of the complexes described herein.

According to the present invention, there is also provided a process of preparing the pharmaceutical composition comprising pemetrexed complex along with pharmaceutically acceptable excipients.

Pharmaceutical compositions of the present invention can comprise pemetrexed disodium:carbohydrate or pemetrexed dipotassium:carbohydrate complex along with one or more pharmaceutically acceptable excipients suitable for parenteral administration.

In one embodiment of the present invention, the pharmaceutical compositions for parenteral administration which are stable and bioavailable.

Further aspect of the present invention also includes administration of the pharmaceutical composition comprising pemetrexed disodium:carbohydrate or pemetrexed dipotassium:carbohydrate complex along with one or more pharmaceutically acceptable excipients in the treatment of cancer, pleural mesothelima and non-small cell lung cancer.

The pharmaceutical composition of the present invention can contain a complex which may be amorphous or crystalline in nature. In a preferred embodiment the pharmaceutical composition comprises a complex is in a crystal form.

The present invention also provides the processes for preparing a pharmaceutical composition comprising a pemetrexed disodium:carbohydrate or pemetrexed dipotassium:carbohydrate complex along with one or more pharmaceutically acceptable excipients which is suitable for parenteral administration.

The term “pharmaceutical composition” includes parenteral dosage forms, such as ready-to-use solutions; lyophilized forms and preparations thereof, like sterile lyophilized powders for intravenous infusion available in single or multi dose vials; aqueous solutions, colloidal nanosuspensions, emulsions, liposomal injections, injectable devices such as, but not limited to, pumps and autoinjectors; sterile powders for injection suitable for solubilization or suspension in liquid prior to injection, liquid dosage forms (liquids, liquid dispersions, suspensions, solutions, emulsions, powders for reconstitution), gels, bolus, depots, implants (rods, capsules, rings) biodegradable or non-biodegradable microparticles/microspheres etc.

Preferably, the pharmaceutical composition comprising pemetrexed disodium:carbohydrate or pemetrexed dipotassium:carbohydrate complex along with one or more pharmaceutically acceptable excipients which is suitable for parenteral administration can be in the form of a ready-to-use dosage form or can be in the form of a lyophilized preparation, which could be reconstituted by mixing with a carrier or a suspending agent before administration.

Further, the pharmaceutical composition comprising pemetrexed disodium:carbohydrate or pemetrexed dipotassium:carbohydrate complex along with one or more pharmaceutically acceptable excipients which is suitable for parenteral use may be administered via intramuscular route, intravenous route, subcutaneous route, intra dermal route, intra peritoneal route and the like.

The pharmaceutical composition comprising pemetrexed disodium:carbohydrate or pemetrexed dipotassium:carbohydrate complex along with one or more pharmaceutically acceptable excipients is not only restricted for parenteral administration, but it may envisage other pharmaceutical dosage forms such as, but not limited to, tablets (single layer, bilayer, multilayer, tablet in tablet) which may be uncoated, film coated, carbohydrate coated, powder coated, enteric coated, seal coated; capsules (filled with powders, powders for reconstitution, pellets, beads, mini-tablets, pills, micro-pellets, small tablet units, multi unit pellet systems (MUPS), disintegrating tablets, dispersible tablets, granules, microspheres and multiparticulates or combinations thereof), sachets (filled with powders, pellets, beads, mini-tablets, pills, micro-pellets, small tablet units, multi unit pellet systems (MUPS), disintegrating tablets, dispersible tablets, granules, microspheres, multiparticulates or combinations thereof) and sprinkles. Other dosage forms such as liquid dosage forms (liquids, liquid dispersions, suspensions, solutions, emulsions, sprays, spot-on), gels, aerosols, ointments, creams, controlled release formulations, delayed release formulations, extended release formulations, pulsatile release formulations, dual release formulations etc. may also be envisaged under the ambit of the invention.

The pharmaceutical composition comprising the pemetrexed disodium:carbohydrate or pemetrexed dipotassium:carbohydrate complex along with one or more pharmaceutically acceptable excipients can be prepared according to techniques known in the art using suitable pharmaceutically acceptable excipients such as, but not limited to bulking agents, pH adjusting agents, carriers or suspending agents, antibacterial preservatives, chelating agents, stabilizers, sequestering agents, antioxidants and tonicity adjusting agents or combinations thereof.

Bulking agents (also called “cryoprotectant/lyoprotectant”) are excipients which are capable of making the pharmaceutical composition of the present invention isotonic with blood at the time of administration.

Suitable bulking agents or diluents, that can be used, in the pharmaceutical composition of the present invention, include, but are not limited to, mannitol, sucrose, maltose, xylitol, glucose, starches, sorbitol, fructose, galactose, ribose, xylose, trehalose, lactose, raffinose, melezitose, glycerol, erythritol, maltitol, lactitol, D & L series of rare carbohydrates, their salts and the like or mixtures thereof.

The pharmaceutical composition of the present invention may further comprise a pH adjusting agent, which is used in an amount to adjust the pH from about 4 to about 10 before lyophilisation. It may be an acid or base depending upon whether the pH of the pharmaceutical composition needs to be raised or lowered to attain the desired value. Thus, when the pH needs to be lowered, an acidic pH adjusting agent, such as hydrochloric acid, tartaric acid, sulphuric acid, citric acid, phosphoric acid, benzoic acid or acetic acid and the like may be used. And when the pH needs to be raised, a basic pH adjusting agent, such as sodium hydroxide, potassium hydroxide, calcium carbonate, sodium carbonate, magnesium carbonate, potassium carbonate, magnesium oxide, magnesium hydroxide, glutamic acid or histidine and the like may be used. Such pH adjusting agents can either be used singly or in a combination.

The pharmaceutical composition of the present invention may further comprise carriers or suspending agents such as, but not limited to, water for injection, Ringer's solution, isotonic sodium chloride solution and the like or combinations thereof

In addition to the aforementioned, carriers or suspending agents may also include, fixed oils, fatty esters or polyols, sodium carboxymethyl cellulose, hydroxypropyl cellulose, carboxymethyl cellulose, hydroxypropylethyl cellulose, hydroxypropylmethyl cellulose, polyvinylpyrrolidone, various polymers, low-molecular-weight oligomers, natural products, and surfactants (including nonionic and ionic surfactants), such as cetyl pyridinium chloride, gelatin, casein, phospholipids, lecithin (phosphatide), phophatidylcholines dextran. glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters such as Tweens, polyethylene glycols, dodecyl trimethyl ammonium bromide, polyoxyethylene stearates, colloidal silicon dioxide, phosphates, sodium dodecylsulfate, hydroxypropyl celluloses, methylcellulose, hydroxyethylcellulose, hydroxypropylcellulose, hydroxypropyl methylcellulose phthalate, noncrystalline cellulose, magnesium aluminum silicate, triethanolamine, polyvinyl alcohol, 4-(1,1,3,3-tetramethylbutyl)-phenol polymer with ethylene oxide and formaldehyde, poloxamers; poloxamines; charged phospholipids such as dimyristoyl phophatidyl glycerol, dioctylsulfosuccinate; n-decyl-D-maltopyranoside; n-dodecyl-D-glucopyranoside; n-dodecyl-D-maltoside; heptanoyl-N-methylglucamide; n-heptyl-glucopyranoside; n-heptyl-D-thioglucoside; n-hexyl-D-glucopyranoside; nonanoyl-N-methylglucamide; n-nonyl-D-glucopyranoside; octanoyl-N-methylglucamide; n-octyl-glucopyranoside; octyl-D-thioglucopyranoside, their salts and the like or mixtures thereof.

Suitable antibacterial preservatives, that may be employed in the pharmaceutical composition of the present invention are phenylmercuric nitrate, thiomersal, benzalkonium chloride, benzethonium chloride, phenol, 4-amino benzoic acid (PABA), cresol and chlorobutanol; chelating agents such as ethylenediamine tetra acetic acid (EDTA), their salts and the like or mixtures thereof.

Suitable tonicity adjusting agents, that may be employed in the pharmaceutical composition of the present invention are sodium chloride, potassium chloride, dextrose, mannitol, sorbitol, sucrose and lactose; and alkaline substances including one or more of salts of alkali and alkaline earth metals such as sodium hydroxide, potassium hydroxide, sodium carbonate, sodium bicarbonate, and sodium phosphates as well as organic amines such as meglumine and tromethamine, hydrochloric acid, tartaric acid, sulphuric acid, citric acid, phosphoric acid, benzoic acid or acetic acid, sodium hydroxide, potassium hydroxide, calcium carbonate, sodium carbonate, magnesium carbonate, potassium carbonate, magnesium oxide, magnesium hydroxide, glycerine, propylene glycol, PEG, glutamic acid, histidine, their salts and the like or combinations thereof.

The pharmaceutical composition of the invention can comprise a bulking agent, pH adjusting agent, a carrier or suspending agent, an antibacterial preservative, a chelating agent, a stabiliser, a sequestering agent, an antioxidant, a tonicity adjusting agent, or any mixtures thereof.

The pharmaceutical composition of the invention can comprise a bulking agent, a carrier, and a pH adjusting agent.

The pharmaceutical composition can comprise a bulking agent comprising sucrose, mannitol and/or sorbitol, and a pH adjusting agent comprising hydrochloric acid and/or sodium hydroxide. According to another aspect, the present invention provides a process for preparing the pharmaceutical composition suitable for parenteral administration.

In one embodiment, the process comprises dissolving the complex as well as bulking agent in a suitable carrier, adjusting the pH and lyophilizing it.

In another embodiment, the process comprises dissolving the complex as well as bulking agent in a suitable carrier and adjusting the pH.

In another embodiment, the process comprises formation of a sterile powder of the complex along with one or more pharmaceutically acceptable excipients.

In an embodiment, the preferred pemetrexed disodium:carbohydrate complexes in the formulation of the present invention are pemetrexed disodium:mannitol complex, pemetrexed disodium:sorbitol complex, pemetrexed disodium:sucrose complex, most preferred complex is pemetrexed disodium:mannitol complex.

In another embodiment, the preferred pemetrexed dipotassium:carbohydrate complexes in the formulation of the present invention are pemetrexed dipotassium:mannitol complex, pemetrexed dipotassium:sorbitol complex, pemetrexed dipotassium:sucrose complex, most preferred complex is pemetrexed dipotassium:mannitol complex.

The present invention also provides methods of filling containers that contain a pharmaceutical composition of the present invention, comprising: a) pre sterilizing one or more containers, b) filling the containers with the pharmaceutical composition optionally in an aseptic environment, c) stoppering and sealing the filled containers and d) terminal sterilization.

The present invention also provides methods of filling containers that contain a pharmaceutical composition of the present invention, comprising: a) pre sterilizing one or more containers and closures, b) filling the containers with the pharmaceutical composition optionally in an aseptic environment, c) lyophilizing the filled containers, d) stoppering and sealing the filled containers and e) terminal sterilization.

The present invention also provides methods of filling containers that contain a pharmaceutical composition of the present invention, comprising: a) pre sterilizing the pharmaceutical composition, one or more containers and the closures, b) filling the containers with the pharmaceutical composition optionally in an aseptic environment, c) optionally lyophilizing the filled containers, and d) stoppering and sealing the filled containers.

Containers can be in the form of small glass vials that are sealed with a suitable stopper/seal and may also be replaced by other primary containers for example, but not limited to, pre-filled syringes. Vials can be for single use or multi use and may include breakable and non-breakable glass containers, breakable plastic containers, miniature screw-top jars and any other type of container typically of a size capable of holding only unit or multi dose of the pharmaceutical composition of the present invention.

The present invention also provides packaging materials for containers and closures such as, but not limited to, high-density polyethylene (HDPE), low-density polyethylene (LDPE) and/or polypropylene and/or glass, glassine foil, polyvinyl chloride, polyvinylidene dichloride, and the like.

Pharmaceutically acceptable stoppers may also be used to seal the vial containing the pharmaceutical composition of the present invention. Some of the stopper materials include silicone rubber, coated stoppers, slotted bromobutyl rubber, and the like.

The present invention provides a method of treating patients with locally advanced or metastatic nonsquammous non-small cell lung cancer after prior chemotherapy or maintenance treatment of patients whose disease has not progressed after four cycles of platinum based first line chemotherapy by administering a pharmaceutical composition of the present invention.

The present invention also provides a method of initial treatment of patients with locally advanced or metastatic nonsquammous non-small cell lung cancer or mesothelioma by administering a pharmaceutical composition of the present invention in combination with cisplatin.

The present invention further provides the use of pharmaceutical compositions of the present invention for the treatment of patients with locally advanced or metastatic nonsquammous non-small cell lung cancer after prior chemotherapy or maintenance treatment of patients whose disease has not progressed after four cycles of platinum based first line chemotherapy.

The present invention further provides the use of pharmaceutical composition of the present invention for the treatment of locally advanced or metastatic nonsquammous non-small cell lung cancer or mesothelima by administering a pharmaceutical composition of the present invention in combination with cisplatin.

The invention is further defined by reference to the following examples. The examples are for illustration purposes only and in no way would limit the scope of the invention.

Examples of preparing the complex of the present invention:—

### Example 1

### Preparation of Pemetrexed Disodium:Mannitol Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added mannitol (22.5 g) at 25-30° C. The reaction mass was further stirred at 25-30° C. for 10 minutes. The clear filtrate was added to acetone (1.14 l) over a period of 1 hour at 27±2° C. Stirring at 25-30° C. was continued for about 2 hours. The precipitated product was collected by filtration and dried in vacuo for 8 hours to yield 47.5 g of a pharmaceutical compound of pemetrexed disodium:mannitol. The content of the pemetrexed disodium is about 65% of the weight.

### Example 2

### Preparation of Pemetrexed Disodium:Mannitol Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added mannitol (22.5 g) at 25-30° C. The reaction mass was further stirred at 25-30° C. for 10 minutes. The clear filtrate was added to methanol (1.1 l) over a period of 1 hour at 27±2° C. Stirring at 25-30° C. was continued for about 2 hours. The precipitated product was collected by filtration and dried in vacuo for 8 hours to yield 45.0 g of a pharmaceutical compound of pemetrexed disodium:mannitol. The content of the pemetrexed disodium is about 65% of the weight.

### Example 3

### Preparation of Pemetrexed Disodium:Mannitol Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added mannitol (22.5 g) at 25-30° C. The reaction mass was further stirred at 25-30° C. for 10 minutes. The clear filtrate was added to isopropyl alcohol (1.1 l) over a period of 1 hour at 27±2° C. Stirring at 25-30° C. was continued for about 2 hours. The precipitated product was collected by filtration and dried in vacuo for 8 hours to yield 45.0 g of a pharmaceutical compound of pemetrexed disodium:mannitol. The content of the pemetrexed disodium is about 65% of the weight.

### Example 4

### Preparation of Pemetrexed Disodium:Sorbitol Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added sorbitol (22.5 g) at 25-30° C. The reaction mass was further stirred at 25-30° C. for 10 minutes. The clear filtrate was added to acetone (1.1 l) over a period of 1 hour at 27±2° C. Stirring at 25-30° C. was continued for about 2 hours. The precipitated product was collected by filtration and dried in vacuo for 8 hours to yield 47.5 g of pemetrexed disodium:sorbitol complex. The content of the pemetrexed disodium is about 85%.

### Example 5

### Preparation of Pemetrexed Disodium:Sucrose Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added sucrose (48 g) at 25-30° C. The reaction mass was further stirred at 25-30° C. for 10 minutes. The clear filtrate was added to acetone (1.1 l) over a period of 1 hour at 27±2° C. Stirring at 25-30° C. was continued for about 2 hours. The precipitated product was collected by filtration and dried in vacuo for 8 hours to yield 47.5 g of pemetrexed disodium:sucrose complex. The content of the pemetrexed disodium is about 52%.

### Example 6

### Preparation of Pemetrexed Disodium:Glucose Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added glucose (22.5 g) at 25-30° C. The reaction mass was further stirred at 25-30° C. for 10 minutes. The clear filtrate was added to acetone (1.1 l) over a period of 1 hour at 27±2° C. Stirring at 25-30° C. was continued for about 2 hours. The precipitated product was collected by filtration and dried in vacuo for 8 hours to yield 47.5 g of pemetrexed disodium:sorbitol complex. The content of the pemetrexed disodium is about 85%.

### Example 7

### Preparation of Pemetrexed Disodium:Fructose Complex

Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added fructose (22.5 g) at 25-30° C. The reaction mass was further stirred at 25-30° C. for 10 minutes. The clear filtrate was added to acetone (1.1 l) over a period of 1 hour at 27±2° C. Stirring at 25-30° C. was continued for about 2 hours. The precipitated product was collected by filtration and dried in vacuo for 8 hours to yield 47.5 g of pemetrexed disodium:sorbitol complex. The content of the pemetrexed disodium is about 85%.

### Example 8

### Preparation of Pemetrexed Disodium:Mannitol Complex

1) Pemetrexed diethyl ester ditosylate salt was added to purified water and sodium hydroxide, stirred for 3 hours at 20-25° C. The reaction mass was filtered and isopropyl alcohol was added to the clear filtrate. The pH was adjusted to 3.0-3.5 by using hydrochloric acid at 23±2° C. The mixture was stirred at 23±2° C. for 90 minutes, the contents were heated to 53±2° C. and maintained for 30 minutes, and then further cooled to 25±2° C. The material was filtered and dried at 48±2° C. The solid was dissolved in dimethyl sulphoxide and precipitated in isopropyl alcohol, filtered and slurried in a mixture of isopropyl alcohol and water. The solid was filtered and dried at 43±2° C. to obtain pemetrexed.

2) Pemetrexed (30 g) was stirred in purified water (210 ml) at 20-25° C. under a nitrogen atmosphere. The pH of the reaction mass was adjusted to 8, with 10% aqueous sodium hydroxide solution. The reaction mass was further stirred at 20-25° C. for 10 minutes and filtered. To the clear filtrate was added mannitol (22.5 g) at 25-30° C. The reaction mass was further stirred at 25-30° C. for 10 minutes. The clear filtrate was added to the acetone (1.14 l) over a period of 1 hour at 27±2° C. Stirring at 25-30° C. was continued for about 2 hours. The precipitated product was collected by filtration and dried in vacuo for 8 hours to yield 47.5 g of complex pemetrexed disodium:mannitol. The content of the pemetrexed disodium is about 60% of the weight.

Examples of preparing the pharmaceutical compositions of the present invention:—

### Example 1

### Lyophilized Powder for Infusion

| | | Strength |
|---|---|---|
| | | 100 mg/vial |
| Sr. No. | Ingredients | (Qty in mg) |
| 1. | Pemetrexed disodium:mannitol | Equivalent to 100 mg |
| | complex | Pemetrexed |
| 2. | Mannitol | *106.00 |
| 3. | Hydrochloric acid | q.s. to pH 6.6 to 7.8 |
| 4. | Sodium Hydroxide | |

Process:
1) The pemetrexed disodium:mannitol complex was dissolved in water.
2) Mannitol was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.
   * The quantity of Mannitol which is added separately in the formulation in the above example is based on the respective content of Mannitol present in the Pemetrexed Disodium complex.

### Example 2

### Lyophilized Powder for Infusion

| | | Strength |
|---|---|---|
| | | 500 mg/vial |
| Sr. No. | Ingredients | (Qty in mg) |
| 1. | Pemetrexed disodium:mannitol | Equivalent to 500 mg |
| | complex | Pemetrexed |
| 2. | Mannitol | *500.00 |
| 3. | Hydrochloric acid | q.s. to pH 6.6 to 7.8 |
| 4. | Sodium Hydroxide | |

### Process:

1) The pemetrexed disodium:mannitol complex was dissolved in water.
2) Mannitol was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.

* The quantity of Mannitol which is added separately in the formulation in the above example is based on the respective content of Mannitol present in the Pemetrexed Disodium complex.

### Example 3

### Lyophilized Powder for Infusion

| | | Strength |
|---|---|---|
| | | 100 mg/vial |
| Sr. No. | Ingredients | (Qty in mg) |
| 1 | Pemetrexed disodium:sorbitol | Equivalent to 100 mg |
| | complex | Pemetrexed |
| 2. | Sorbitol | *115.5 |
| 3. | Hydrochloric acid | q.s. to pH 6.6 to 7.8 |
| 4. | Sodium Hydroxide | |

### Process:

1) The pemetrexed disodium:sorbitol complex was dissolved in water.
2) Sorbitol was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.

* The quantity of Sorbitol which is added separately in the formulation in the above example is based on the respective content of Sorbitol present in the Pemetrexed Disodium complex.

### Example 4

### Lyophilized Powder for Infusion

| | | Strength |
|---|---|---|
| | | 500 mg/vial |
| Sr. No. | Ingredients | (Qty in mg) |
| 1 | Pemetrexed disodium: sorbitol | Equivalent to 500 mg |
| | complex | Pemetrexed |
| 2. | Sorbitol | *577.5 |
| 3. | Hydrochloric acid | q.s. to pH 6.6 to 7.8 |
| 4. | Sodium Hydroxide | |

### Process:

1) The pemetrexed disodium:Sorbitol complex was dissolved in water.
2) Sorbitol was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.

* The quantity of Sorbitol which is added separately in the formulation in the above example is based on the respective content of Sorbitol present in the Pemetrexed Disodium complex.

### Example 5

### Lyophilized Powder for Infusion

| | | Strength |
|---|---|---|
| | | 100 mg/vial |
| Sr. No. | Ingredients | (Qty in mg) |
| 1 | Pemetrexed disodium: sucrose | Equivalent to 100 mg |
| | complex | Pemetrexed |
| 2. | Sucrose | *195.00 |
| 3. | Hydrochloric acid | q.s. to pH 6.6 to 7.8 |
| 4. | Sodium Hydroxide | |

### Process:

1) The pemetrexed disodium:sucrose complex was dissolved in water.
2) Sucrose was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.

* The quantity of Sucrose which is added separately in the formulation in the above example is based on the respective content of Sucrose present in the Pemetrexed Disodium complex.

### Example 6

### Lyophilized Powder for Infusion

| | | Strength |
|---|---|---|
| | | 500 mg/vial |
| Sr. No. | Ingredients | (Qty in mg) |
| 1 | Pemetrexed disodium: sucrose | Equivalent to 500 mg |
| | complex | Pemetrexed |
| 2. | Sucrose | *920.00 |
| 3. | Hydrochloric acid | q.s. to pH 6.6 to 7.8 |
| 4. | Sodium Hydroxide | |

### Process:

1) The pemetrexed disodium:sucrose complex was dissolved in water.
2) Sucrose was dissolved in the solution obtained in step (1).
3) The pH was adjusted with sodium hydroxide and/or hydrochloric acid.
4) The final volume of the solution was made up with water
5) The vials were filled with the solution obtained in step (4) and lyophilized.

* The quantity of Sucrose which is added separately in the formulation in the above example is based on the respective content of Sucrose present in the Pemetrexed Disodium complex.

It will be appreciated that the invention may be modified within the scope of the appended claims.

XRD measurements were made using the following settings:

SCAN: 3.0/40.0/0.02/0.6 (sec), Cu (40 kV, 30 mA), I(max)=2369.

PEAK: 15-pts/Parabolic Filter, Threshold=0.0, Cutoff=0.1%, BG=1/0.7, Peak-Top=Summit.

NOTE: Intensity=Counts, 2T(0)=0.0 (deg), Wavelength to compute d-spacing=1.54056 Å (Cu/K-alpha1).

1. A complex of pemetrexed with a co-former.

2. A complex according to claim 1, wherein the pemetrexed is in the form of a pharmaceutically acceptable derivative, which optionally comprises pharmaceutically acceptable salts, solvates, hydrates, isomers, esters, tautomers, anhydrates, enantiomers, complexes, polymorphs or prodrugs.

3. A complex according to claim 2, wherein the pemetrexed is in the form of pemetrexed disodium or pemetrexed dipotassium.

4. A complex according to claim 1, wherein the complex comprises pemetrexed disodium or pemetrexed dipotassium and a carbohydrate.

5. A complex according to claim 4, wherein the carbohydrate comprises a sugar and/or sugar alcohol.

6. A complex according to claim 4, wherein the carbohydrate comprises glucose, dextrose, fructose, galactose, ribose, sucrose, xylose, trehalose, lactose, maltose, raffinose, melezitose, glycerol, mannitol, sorbitol, erythritol, maltitol, lactitol, and/or D series or L series rare sugars.

7. A complex according to claim 1, wherein the pemetrexed complex comprises at least one or more of the following complexes: pemetrexed disodium:mannitol complex, pemetrexed disodium:sorbitol complex, pemetrexed disodium:sucrose complex, pemetrexed disodium:glucose complex, or pemetrexed disodium:fructose complex.

8. A complex according to claim 1, wherein the pemetrexed complex comprises at least one or more of the following complexes: pemetrexed dipotassium:mannitol complex, pemetrexed dipotassium:sorbitol complex, pemetrexed dipotassium:sucrose complex, pemetrexed dipotassium:glucose complex, or pemetrexed dipotassium:fructose complex.

9. A complex according to claim 1, wherein the pemetrexed complex is in crystal form.

10. A complex according to claim 4, wherein the pemetrexed disodium or dipotassium and carbohydrate present in the complex are present in a stoichiometric ratio.

11. A complex according to claim 1, wherein the complex comprises pemetrexed disodium or dipotassium and mannitol, wherein mannitol is present in the complex in an amount from about 20% to about 50% by weight of the complex, optionally from about 30% to about 40% by weight of the complex.

12. A complex according to claim 1, wherein the complex comprises pemetrexed disodium and mannitol in a crystalline form with an XRD pattern comprising peaks at 9.619, 20.299 and 22.82 °2θ±0.2°2θ, optionally further comprising peaks at 15.44, 20.98, 21.98, 23.8, 24.559, 25.16 and 36.00 2θ±0.2 °2θ, and optionally further comprising peaks at 4.66, 10.38, 14.12, 15.719, 17.14, 17.90, 18.559, 19.261, 25.861, 26.90, 27.78 and 28.82 °2θ±0.2 °2θ.

13. A complex according to claim 1, wherein the complex comprises pemetrexed disodium and mannitol characterised by an X-ray powder diffraction spectrum as shown in FIG. 1.

14. A process for the preparation of a complex according to claim 1, wherein the process comprises;
(1) dispersing pemetrexed into an aqueous solvent to form a solution;
and adjusting the pH of the solution;
(2) optionally removing insolubles by filtration;
(3) adding a carbohydrate to the filtrate;
(4) precipitating and drying the complex from the solution.

15. A process according to claim 14, wherein the pemetrexed starting material is in the form of a salt, a free acid, any polymorphic form or a mixture of any polymorphic forms, in crude form, in anhydrous form, or in a hydrated or solvated form.

16. A process according to claim 14, wherein the process comprises adjusting the pH of the solution to from 7.8 to 8.3.

17. A process according to claim 14, wherein the process comprises adjusting the pH with sodium hydroxide.

18. A process according to claim 14, wherein the reaction is carried out at a temperature ranging from about 20° C. to about 30° C.

19. A process according to claim 14, wherein the step of precipitation comprises the addition of an antisolvent.

20. A process according to claim 14, wherein the anti-solvent is selected from the group consisting of acetone, C1-6 alcohols and acetonitrile.

21. (canceled)

22. (canceled)

23. (canceled)

24. A method of treating a subject with cancer, pleural mesothelima and/or non-small cell lung cancer, wherein the method comprises administering a complex according to claim 1 to a patient in need thereof.

25. A pharmaceutical composition comprising a complex of pemetrexed with a co-former, and one or more pharmaceutically acceptable excipients.

26. A pharmaceutical composition according to claim 25, wherein the pemetrexed is in the form of a pharmaceutically acceptable derivative, which optionally comprises pharmaceutically acceptable salts, solvates, hydrates, isomers, esters, tautomers, anhydrates, enantiomers, complexes, polymorphs or prodrugs.

27. A pharmaceutical composition according to claim 25, wherein the pemetrexed complex comprises pemetrexed disodium or pemetrexed dipotassium.

28. A pharmaceutical composition according to claim 25, wherein the pemetrexed complex comprises pemetrexed disodium or pemetrexed dipotassium and a co-crystal forming agent.

29. A pharmaceutical composition according to claim 25, wherein the pemetrexed complex comprises pemetrexed disodium or pemetrexed dipotassium and a carbohydrate.

30. A pharmaceutical composition according to claim 29, wherein the carbohydrate comprises a sugar and/or sugar alcohol.

31. A pharmaceutical composition according to claim 29, wherein the carbohydrate comprises glucose, dextrose, fructose, galactose, ribose, sucrose, xylose, trehalose, lactose, maltose, raffinose, melezitose, glycerol, mannitol, sorbitol, erythritol, maltitol, lactitol, and/or D series or L series rare sugars.

32. A pharmaceutical composition according to claim 25, wherein the pemetrexed complex comprises at least one or more of the following combinations: pemetrexed disodium:mannitol complex, pemetrexed disodium:sorbitol complex, pemetrexed disodium:sucrose complex, pemetrexed disodium:glucose complex, pemetrexed disodium:fructose complex; most preferred combination is pemetrexed disodium:mannitol complex.

33. A pharmaceutical composition according to claim 25, wherein the pemetrexed complex comprises at least one or more of the following combinations: pemetrexed dipotassium:mannitol complex, pemetrexed dipotassium:sorbitol complex, pemetrexed dipotassium:sucrose complex, pemetrexed dipotassium:glucose complex, pemetrexed dipotassium:fructose complex; most preferred combination is pemetrexed dipotassium:mannitol complex.

34. A pharmaceutical composition according to claim 25, wherein the pemetrexed complex is in crystal form.

35. A pharmaceutical composition according to claim 29, wherein the pemetrexed disodium or pemetrexed dipotassium and carbohydrate present in the complex are present in a stoichiometric ratio.

36. (canceled)

37. A pharmaceutical composition according to claim 25, wherein the composition is in the form of ready-to-use solutions;
lyophilized forms and preparations thereof, sterile lyophilized powders for intravenous infusion available in single or multi dose vials; aqueous solutions, liquid dispersions, suspensions, colloidal nanosuspensions, emulsions, liposomal injections, gels, bolus, depots, implants (rods, capsules, rings), injectable devices such as pumps and autoinjectors; and sterile powders for injection suitable for solubilization or suspension in liquid prior to injection.

38. (canceled)

39. (canceled)

40. (canceled)

41. (canceled)

42. (canceled)

43. (canceled)

44. (canceled)

45. (canceled)

46. (canceled)

47. (canceled)

48. A pharmaceutical composition according to claim 49, wherein the bulking agent comprises sucrose, mannitol and/or sorbitol, and the pH adjusting agent comprises hydrochloric acid and/or sodium hydroxide.

49. A pharmaceutical composition according to claim 25, wherein the at least one or more pharmaceutically acceptable excipients comprise a bulking agent, pH adjusting agent, a carrier or suspending agent, an antibacterial preservative, a chelating agent, a stabiliser, a sequestering agent, an antioxidant, a tonicity adjusting agent, or any combination thereof.

50. A pharmaceutical composition according to claim 25, wherein the composition has a pH from 4 to 10, preferably from 6 to 8.5.

51. (canceled)

52. (canceled)

53. (canceled)

54. A method of treating a subject with cancer, pleural mesothelima and/or non-small cell lung cancer, wherein the method comprises administering a pharmaceutical composition according to claim 25, optionally in combination with cisplatin to a patient in need thereof.

55. (canceled)

56. A process of preparing a pharmaceutical composition according to claim 25, wherein the process comprises dispersing a complex of pemetrexed optionally with at least one or more pharmaceutically acceptable excipients, adjusting the pH using a suitable pH adjusting agent; optionally filling the mixture into a container; and optionally lyophilising the mixture.

57. (canceled)

58. (canceled)
